(19) United States
(12) Patent Application Publication
(10) Pub. No.: US 2026/0085042 A1
(43) Pub. Date: Mar. 26, 2026

(54) METHOD FOR PREPARING INDIGO OR A SUBSTITUTED DERIVATIVE THEREOF

(71) Applicant: PILI, Toulouse Cedex 4 (FR)

(72) Inventors: Filipe GOMES, Toulouse Cedex 4 (FR); Océane CHEVRON, Toulouse Cedex 4 (FR); Kevin COTTET, Toulouse Cedex 4 (FR); Guillaume BOISSONNAT; Debora VILONA, Chassieu (FR); Cyril BOSSET, Chassieu (FR); Vivien HENRYON, Chassieu (FR)

(21) Appl. No.: 18/875,479

(22) PCT Filed: Jun. 16, 2023
(86) PCT No.: PCT/FR2023/050875

§ 371 Date: Dec. 16, 2024

(30) Foreign Application Priority Data
Jun. 16, 2022 (FR) FR2205875

(51) Int. Cl. C07D 209/36

(52) U.S. Cl. CPC C07D 209/36

(57) ABSTRACT

A method for the synthesis of dyes is described. More particularly, a method is described for preparing indigo, optionally substituted, that includes reacting anthranilic acid, optionally substituted, with glyoxylic acid under hydrogenation in the presence of a metal catalyst in a solvent, to obtain 2-(carboxymethylamino)benzoic acid, optionally substituted, and conversion of the latter to indigo, optionally substituted.

### SUBJECT MATTER OF THE INVENTION

The present invention relates to the synthesis of dyes. More particularly, the present invention relates to a method of preparing indigo, optionally substituted, starting from an anthranilic acid, optionally substituted.

### BACKGROUND OF THE INVENTION

With its strong, deep blue colour, which we find in particular in denim and in Tuareg scarves, indigo has been known for millennia. Historically, indigo was mainly obtained by extraction and then oxidation of indican contained in plants of the family Indigofera, in particular the indigo plant. In the 17th and 18th centuries, to meet the strong demand for indigo in the West, plantations of indigo plant were established in North America, in the French Antilles and in India. However, production of natural indigo has gradually been replaced by production by chemical synthesis. Today, 50 000 tonnes of synthetic indigo are produced each year and the major part of this output is intended for dyeing the 4 billion articles of denim clothing manufactured each year.

Since the first synthesis carried out at the end of the 19th century by the chemist Adolf von Baeyer, starting from 2-nitrobenzaldehyde and acetone, several methods of preparing indigo have been proposed.

Karl Heumann (Chem. Ber, 1890, 3431) proposed two strategies for synthesis of indigo. A first strategy, in which aniline and chloroacetic acid react to form N-phenylglycine, which in its turn is converted to indoxyl, in a basic medium. The indoxyl is then oxidized to form indigo. However, low yields are obtained with this first strategy. The second strategy uses anthranilic acid instead of aniline: it is reacted with chloroacetic acid to produce 2-(carboxymethylamino)benzoic acid. The latter is then brought in contact with a base, to form 2-indoxylcarboxylic acid. Indigo is finally produced by heating and oxidizing said acid. However, the yields obtained are still modest, and the method is very energy-consuming, is not environmentally friendly, and uses toxic reactants, in particular chloroacetic acid.

The formation of 2-(carboxymethylamino)benzoic acid starting from anthranilic acid and chloroacetic acid is a key step in the synthesis of indigo. Various reaction conditions have been described, but the yields obtained are not high enough for an industrial process. For example, the reaction described in patent application US2008/051426 is carried out in the presence of sodium bicarbonate at 40-45° C. and gives a yield of only 80%. Lai et al. (Helvetica Chimica Acta 2008, 91, 1975-1983) describe the same reaction in the presence of a mixture of sodium bicarbonate and sodium hydroxide, carried out with reflux of the water. A yield of 2-(carboxymethylamino)benzoic acid of 90% is obtained. Besides the insufficient yields, the chloroacetic acid used in these methods is a toxic reactant.

Koeppe et al. (ChemPhotoChem 2019, 3, 613-618) describe the formation of 2-(carboxymethylamino)benzoic acid by reductive amination from anthranilic acid and ethyl glyoxylate, in the presence of sodium cyanoborohydride. However, this reaction generates a lot of waste, in particular because of the sodium cyanoborohydride, which is used in large amounts and is not recyclable. Furthermore, the use of a glyoxylate necessitates an additional step of saponification with sodium hydroxide to obtain said acid.

Thus, there is therefore still a real need for an improved method of preparing indigo that is simple, efficient, and environmentally friendly.

### SUMMARY OF THE INVENTION

In this context, the inventors have developed a method for preparing indigo, optionally substituted, in three steps, which is simple, and suitable for application on an industrial scale, carried out starting from anthranilic acid, optionally substituted, and glyoxylic acid. The key step of this method uses a metal catalyst, in particular a palladium catalyst, advantageously heterogeneous, under a hydrogen atmosphere, giving 2-(carboxymethylamino)benzoic acid, optionally substituted, with excellent yields. This acid may then be converted efficiently to indigo, optionally substituted, in particular via a cyclization in the presence of acetic anhydride and a dimerization in a basic medium. The method is very environmentally friendly, insofar as it generates little waste, and uses solvents and a catalyst that can be recycled. Furthermore, the method uses glyoxylic acid, which is non-toxic and can be obtained from ethanol or biosourced glycolic acid.

Thus, the present invention relates to a method for preparing a compound of formula (III):

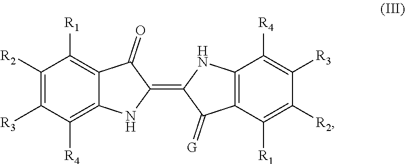

in which each of R₁, R₂, R₃ and R₄ is independently a hydrogen, a halogen, —CN, —NO₂, —C(O)H, —SO₃H, —CO₂H, —SO₃⁻M₁⁺, —CO₂⁻M₂⁺, —SO₃R₅, —CO₂R₆, —C(O) R₇ or —OR₈,
each of M₁⁺ and M₂⁺ being independently a cation,
each of R₅, R₆, R₇, and R₈ being independently a C₁-C₆ aliphatic group or an aryl; said method comprising the following steps:
a) reacting a compound of formula (I):

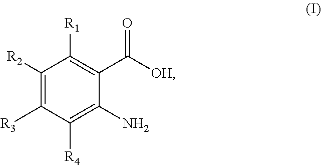

in which each of R₁, R₂, R₃ and R₄ is as defined above, with glyoxylic acid under hydrogenation in the presence of a metal catalyst in a solvent, to obtain a compound of formula (II):

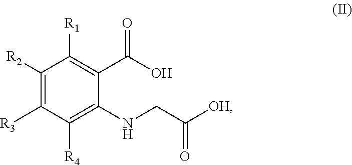

in which each of R₁, R₂, R₃ and R₄ is as defined above;
b) conversion of said compound of formula (II) to said compound of formula (III);
c) recovery of said compound of formula (III).

In a preferred embodiment, R₁, R₂, R₃, and R₄ are hydrogens.

Preferably, the metal catalyst is a palladium, nickel, or platinum catalyst. More preferably, the metal catalyst is palladium on carbon, palladium on aluminium oxide, nickel on aluminium oxide, nickel on aluminium oxide and silica, or platinum on carbon. Even more preferably, the metal catalyst is palladium on carbon.

In a particular embodiment, the method according to the invention further comprises a step of recovery of the metal catalyst, after step a), preferably by filtration.

In another particular embodiment, the method according to the invention further comprises, after step a), a step of recovery of the solvent from step a), preferably by distillation or evaporation at reduced pressure.

Preferably, the solvent from step a) is a polar solvent, such as THF or a THF/water mixture.

According to another particular embodiment, glyoxylic acid is obtained from ethanol or from biosourced glycolic acid.

According to another particular embodiment, the reaction in step a) is carried out at a temperature between 35° C. and 120° C., preferably for a time between 30 seconds and 8 h.

In particular, the amount of metal catalyst in step a) is between 0.0001% and 40% by weight, preferably between 5% and 25% by weight, relative to the weight of the compound of formula (I), and the hydrogen pressure is between 1 and 30 bar, preferably between 5 and 20 bar.

In particular, step b) comprises:
b1) reacting a compound of formula (II) as defined in the present application with acetic anhydride, to obtain a compound of formula (II′):

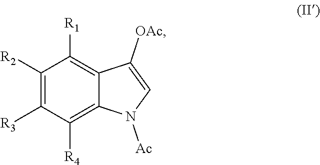

in which each of R₁, R₂, R₃ and R₄ is independently a hydrogen, a halogen, —CN, —NO₂, —C(O)H, —SO₃H, —CO₂H, —SOM₁^{t}, —CO₂⁻M₂⁺, —SO₃R₅, —CO₂R₆, —C(O) R₇ or —OR₈,
each of M₁⁺ and M₂⁺ being independently a cation,
each of R₅, R₆, R₇, and R₈ being independently a C₁-C₆ aliphatic group or an aryl; and
b2) reacting said compound of formula (II′) with a base, to obtain a compound of formula (III) as defined in the present application.

According to a preferred embodiment, the method according to the invention comprises the following steps:
a) reacting a compound of formula (I) as defined in the present application with glyoxylic acid in the presence of palladium on carbon and under a hydrogen atmosphere, in a polar solvent, at a temperature between 35° C. and 120° C., preferably for a time between 30 seconds and 8 h, to obtain a compound of formula (II) as defined in the present application:
b1) reacting said compound of formula (II) with acetic anhydride, in the presence of a base, preferably an amine base, such as triethylamine, said reaction being carried out successively:
at a temperature between 10° C. and 40° C., preferably for a time between 15 minutes and 3 hours, and then
at a temperature between 70° C. and 110° C., preferably for a time between 15 minutes and 3 hours, to obtain a compound of formula (II′) as defined in the present application:
b2) reacting said compound of formula (II′) with a base, preferably a hydroxide, such as sodium hydroxide, in water, at a temperature between 80° C. and 110° C., preferably for a time between 1 h and 5 h, to obtain a compound of formula (III) as defined in the present application; and
c) recovery of said compound of formula (III).

In a particular embodiment, the compound of formula (I), in particular anthranilic acid, is biosourced. In a more particular embodiment, the compound of formula (I), in particular anthranilic acid, is produced by a recombinant host cell, said cell preferably being microbial. Preferably, the recombinant microbial host cell is selected from Escherichia (Escherichia coli), Streptomyces, Bacillus, Cupridavidus, Corynebacterium, Mycobacterium, Kitasatospora, Luteipulveratus, Thermobifida, Thermomonospora, Frankia, Pseudonocardia, Saccharothrix, Kutzneria, Lentzea, Prauserella, Salinispora, Micromonospora, Actinoplanes, Catenulispora, Mycolicibacterium, Dietzia, Aeromicrobium, Nonomuraea, Blastococcus, Modestobacter, Saccharopolyspora, Amycolatopsis, Actinopolyspora, Acidimicrobium, Photorhabdus, Hoeflea, Azospirillum, Crinalium, and Cylindrospermum, preferably selected from Escherichia, Streptomyces, Corynebacterium and Bacillus, and even more preferably, the recombinant microbial host cell is Escherichia coli.

### DETAILED DESCRIPTION

### Definitions

The expression “Cₓ-C_{y}” associated with a chemical group, in which x and y are integers, signifies that said chemical group comprises from x to y carbon atoms. For example, if the expression C₁-C₆ is associated with a chemical group, this signifies that said chemical group comprises from 1 to 6 carbon atoms, in particular 1, 2, 3, 4, 5, or 6 carbon atoms.

“Aliphatic group” means a linear or branched, saturated or unsaturated, cyclic or acyclic (preferably acyclic) hydrocarbon group. In a particular embodiment, said C₁-C₆ aliphatic group is a C₁-C₆ alkyl, a C₂-C₆ alkenyl, or a C₂-C₆ alkynyl.

“Alkyl” means a saturated, acyclic, linear or branched hydrocarbon group. Examples of alkyl (or C₁-C₆ alkyl) are in particular a methyl, ethyl, propyl, isopropyl, butyl, isobutyl, tert-butyl, pentyl, or hexyl.

“Alkenyl” means an unsaturated, linear or branched acyclic hydrocarbon group, comprising at least one carbon-carbon double bond. Examples of alkenyl (or C₂-C₆ alkenyl) are in particular an ethenyl, propenyl, isopropenyl, butenyl, isobutenyl, pentenyl, or hexenyl.

“Alkynyl” means an unsaturated, linear or branched acyclic hydrocarbon group, comprising at least one carbon-carbon triple bond. Examples of alkynyl (or C₂-C₆ alkynyl) are in particular an ethynyl, propynyl, butynyl, pentynyl, or hexynyl.

“Aryl” means a mono- or polycyclic aromatic hydrocarbon group, preferably having from 6 to 20 ring members. Examples of aryl groups are phenyl, biphenyl, and naphthyl, preferably a phenyl.

“Halogen” means a fluorine, a chlorine, a bromine or an iodine, preferably a bromine.

“Cation” means an atom or group of atoms having a positive charge (for example, a charge of +1, +2, +3, or +4). Said cation may be an organic or inorganic cation, preferably an inorganic cation. Examples of organic cations are in particular a tetra(C₁-C₆ alkyl) ammonium cation, a pyridinium cation, or a tetra(C₁-C₆ alkyl)phosphonium cation. Examples of inorganic cations are in particular an alkaline cation (such as a sodium, a lithium, a potassium, or a caesium), an alkaline-earth cation (such as a magnesium or a calcium), an aluminium cation, or an ammonium cation (NH₄⁺). A preferred cation is a sodium cation.

In the present application, the abbreviation “Ac” signifies “acetyl” (i.e. —C(O)—CH₃).

In the present application, the term “about” associated with a value is a term that is familiar to a person skilled in the art and signifies that said value may vary to a certain extent depending on the context in which the term is used. If certain uses of this term are not clear to a person skilled in the art depending on the context, then “about” signifies more or less 30%, more or less 20%, preferably more or less 10% of said associated value.

Unless stated otherwise, when a range is expressed by means of the expression “between”, the limit values are included within the range described.

The present invention provides a method that makes it possible to obtain a compound of the indigo type, optionally substituted, in a manner that is simple, efficient, and environmentally friendly.

The method according to the invention is a method for preparing a compound of formula (III):

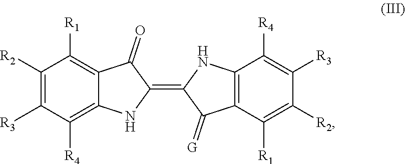

said method comprising the following steps:
a) reacting a compound of formula (I):

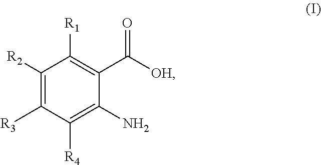

with glyoxylic acid under hydrogenation in the presence of a metal catalyst in a solvent, to obtain a compound of formula (II):

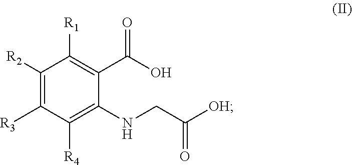

b) conversion of said compound of formula (II) to a compound of formula (III) as defined above:
c) recovery of said compound of formula (III);
where, in these formulae, each of R₁, R₂, R₃ and R₄ is independently a hydrogen, a halogen, —CN, —NO₂, —C(O)H, —SO₃H, —CO₂H, —SO₃M₁⁺, —CO₂M₂⁺, —SO₃R₅, —CO₂R₆, —C(O) R₇ or —OR₈,
each of M₁⁺ and M₂⁺ being independently a cation (preferably an alkaline cation such as sodium),
each of R₅, R₆, R₇, and R₈ being independently a C₁-C₆ aliphatic group (such as a C₁-C₆ alkyl) or an aryl (such as a phenyl).

In a particular embodiment, the compounds of formula (I), (II), and (III) are such that at least two groups (preferably, at least three) out of R₁, R₂, R₃, and R₄ are hydrogens. In a particular embodiment, the compounds of formula (I), (II), and (III) are such that R₁, R₃, and R₄ are hydrogens, and R₂ is —SO₃⁻Na⁺.

In another particular embodiment, the compounds of formula (I), (II), and (III) are such that each of R₁, R₂, R₃ and R₄ is independently a hydrogen or a bromine, provided that at least one of R₁, R₂, R₃, and R₄ is a hydrogen and at least one other of R₁, R₂, R₃, and R₄ is a bromine. In a preferred embodiment, the compounds of formula (I), (II), and (III) are such that R₁, R₂, R₃, and R₄ are hydrogens. In said preferred embodiment, the compound of formula (I) is anthranilic acid, the compound of formula (II) is 2-(carboxymethylamino)benzoic acid, and the compound of formula (III) is indigo. Indigo (CAS No.: 482-89-3) is also called indigotin and is represented by the following formula:

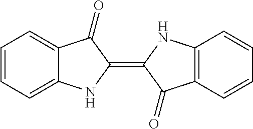 [SMILES: O=C1/C(=C2\Nc3ccccc3C2=O)Nc2ccccc21]

Step a) of the method according to the invention comprises reacting a compound of formula (I) as defined in the present application with glyoxylic acid in a solvent, under hydrogenation in the presence of a metal catalyst (or equivalently “under metal-catalysed hydrogenation”). The metal-catalysed hydrogenation in step a) allows reduction of the imine formed by coupling between the amine function of said compound of formula (I) and the aldehyde function of glyoxylic acid, thus forming a compound of formula (II) as defined in the present application.

The metal-catalysed hydrogenation uses a metal catalyst, in a hydrogen atmosphere. The hydrogen pressure in step a) may in particular be between 1 and 30 bar, preferably between 5 and 20 bar, better still about 10 bar.

The metal catalyst is advantageously heterogeneous. The term “heterogeneous” qualifying a catalyst is familiar to a person skilled in the art and signifies that the catalyst is not in the same phase as the reaction mixture in which it is applied. Typically, a heterogeneous metal catalyst used in step a) is solid and the reaction mixture is liquid and/or gaseous.

The metal catalyst is advantageously a transition metal catalyst. Examples of transition metals are in particular nickel, palladium, platinum, rhodium, or a combination thereof. In a particular embodiment, the metal catalyst is a palladium, nickel, or platinum catalyst. Preferably, the metal catalyst is palladium on carbon, palladium on aluminium oxide, nickel on aluminium oxide, nickel on aluminium oxide and silica, or platinum on carbon. As an example of nickel on aluminium oxide and silica, we may mention the NiSat® range, including Ni/Al₂O₃—SiO₂ 54% Ni-310RS, marketed by the company Clariant. As an example of nickel on aluminium oxide, we may mention Ni/Al₂O₃21% Ni (HTC 500Ni—Johnson-Matthey).

Even more preferably, the metal catalyst is palladium on carbon.

The amount of metal catalyst used in step a) may be between 0.0001% and 40% by weight, in particular between 1% and 30% by weight, preferably between 5% and 25% by weight, better still between 5% and 15% by weight, for example about 10% by weight, relative to the weight of said compound of formula (I).

The reaction in step a) may be carried out at a temperature between 35° C. and 120° C., in particular between 35° C. and 90° C., or even between 35° C. and 65° C., for example about 50° C.

The reaction in step a) may be carried out for a time between 30 seconds and 15 h, in particular between 15 minutes and 8 h, or even between 2 h and 8 h, for example about 4 h. The solvent in step a) is in particular a polar solvent, for example an ether such as THE or diethyl ether, an alcohol such as ethanol, propanol or butanol, dimethylsulphoxide, water, or a combination thereof, preferably tetrahydrofuran (THF) or a THF/water mixture. Other solvents, in particular those used conventionally in metal-catalysed hydrogenation reactions, may also be used.

In a particular embodiment, step a) comprises:
i) forming a mixture by adding a compound of formula (I) as defined in the present application and the metal catalyst, preferably palladium on carbon, to a polar solvent, such as THE or a THF/water mixture, under stirring:
ii) placing the mixture from step i) under hydrogen pressure, said pressure preferably being between 1 and 30 bar (better still between 5 and 20 bar):
iii) heating the mixture from step ii) at a temperature between 35° C. and 120° C. (in particular, between 35° C. and 65° C.); and
iv) adding glyoxylic acid to the mixture from step iii).

Preferably, the reaction in step a) is carried out for a time between 30 seconds and 8 h, better still between 15 minutes and 8 h, or even between 2 h and 8 h, for example about 4 h.

The glyoxylic acid used as raw material in the method of the invention may be produced by any type of method of chemical synthesis. Preferably, the glyoxylic acid is obtained from biosourced ethanol (also called “bioethanol”) or from biosourced glycolic acid.

The compound of formula (I), used as raw material in the method of the invention, may be produced by a method of chemical synthesis or a biological method. Preferably, the compound of formula (I) (preferably anthranilic acid) is obtained by a biological method, for example by means of a host cell or a genetic sequence thereof.

In a preferred embodiment, the compound of formula (I) (preferably anthranilic acid) is produced by a recombinant host cell, said cell preferably being microbial. The use of said recombinant host cell makes it possible to supply the compound of formula (I) (preferably anthranilic acid) in large amounts and in mild conditions by simple fermentation. The term “recombinant host cell” denotes a cell that does not exist in nature and that contains a modified genome resulting either from a deletion, or from an insertion, or from a modification of one or more genetic elements. The term “host cell” also includes any descendant of a parent host cell that is not identical to the parent host cell on account of mutations that arise during replication. The host cell may be a microbial or plant host cell. Preferably, the host cell is a microbial host cell. As used herein, the term “microbial host cell” denotes a bacterium, a filamentous fungus or a yeast, preferably a bacterium or a yeast, more preferably a bacterium.

In particular, the host cell may be a bacterium of the genus Escherichia, Streptomyces, Corynebacterium, Bacillus, Achromobacter, Brevibacterium, Arthrobacter, Flavobacterium, or Pseudomonas. More particularly, the host cell may be a bacterium Escherichia coli, Streptomyces coelicolor, Bacillus subtilis, Bacillus megaterium, Achromobacter-polymorph, Achromobacter xerosis, Brevibacterium protoformiae, Arthrobacter oxydans, Flavobacterium esteraromaticum, Pseudomonas fluorescens, Pseudomonas diminuta, or Pseudomonas aeruginosa.

Preferably, the host cell is a bacterium of the genus Escherichia, such as Escherichia coli or Streptomyces, such as Streptomyces coelicolor. The biological methods of production of anthranilic acid starting from a recombinant microbial host cell formed the subject matter of International patent application No. PCT/EP2021/079927, to which a person skilled in the art will easily be able to refer.

A metal catalyst, heterogeneous in particular, offers the advantage of being able to be separated more easily from the reaction mixture in step a), so as then to be able to be recovered and reused, i.e. recycled. In a particular embodiment, the method according to the invention further comprises, after step a), a step of recovery of the metal catalyst, in particular heterogeneous. This step of recovery of the catalyst is preferably carried out by filtration, for example by filtration on Celite.

The solvent used in step a) may also be recovered and reused, i.e. recycled. In a particular embodiment, the method according to the invention further comprises, after step a), a step of recovery of said solvent. This solvent recovery step is preferably carried out by distillation or evaporation at reduced pressure.

Step b) of the method of the invention comprises conversion of a compound of formula (II) as defined in the present application to a compound of formula (III) as defined in the present application.

Preferably, step b) comprises:
b1) reacting a compound of formula (II) as defined in the present application with acetic anhydride, to obtain a compound of formula (II′):

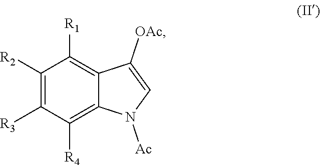

in which each of R₁, R₂, R₃ and R₄ is independently a hydrogen, a halogen, —CN, —NO₂, —C(O)H, —SO₃H, —CO₂H, —SO₃M₂⁺, —CO₂M₂*, —SO₃R₅, —CO₂R₆, —C(O) R₇ or —OR₈,
each of M₁⁺ and M₂⁺ being independently a cation (preferably an alkaline cation such as sodium),
each of R₅, R₆, R₇, and R₈ being independently a C₁-C₆ aliphatic group (such as a C₁-C₆ alkyl) or an aryl (such as a phenyl); and
b2) reacting said compound of formula (II′) with a base, to obtain a compound of formula (III) as defined in the present application.

In a particular embodiment, the compound of formula (II′) is such that at least two groups (preferably, at least three) out of R₁, R₂, R₃, and R₄ are hydrogens.

In a particular embodiment, the compound of formula (II′) is such that R₁, R₃, and R₄ are hydrogens, and R₂ is −SO₃Na⁺.

In another particular embodiment, the compound of formula (II′) is such that each of R₁, R₂, R₃ and R₄ is independently a hydrogen or a bromine, provided that at least one of R₁, R₂, R₃, and R₄ is a hydrogen and at least one other of R₁, R₂, R₃, and R₄ is a bromine.

In a preferred embodiment, the compound of formula (II′) is such that R₁, R₂, R₃, and R₄ are hydrogens. In said preferred embodiment, the compound of formula (II′) is 1-acetylindol-3-yl acetate.

Preferably, the reaction in step b1) is carried out in the presence of a base. When it is present, said base is advantageously an amine base. Examples of amine bases are in particular triethylamine, diisopropylethylamine, dimethylphenylamine, or piperidine.

Preferably, said amine base is triethylamine.

In a particular embodiment, step b1) comprises formation of a mixture comprising (preferably, consisting of) a compound of formula (II), acetic anhydride and the optional base (preferably triethylamine), and stirring the resultant reaction mixture.

The reacting in step b1) is advantageously carried out successively:
at a temperature between 10° C. and 40° C., preferably for a time between 15 minutes and 3 hours (for example, about 30 minutes), and then
at a temperature between 70° C. and 110° C., preferably for a time between 15 minutes and 3 hours (for example, about 30 minutes).

The molar amount of acetic anhydride in step b1) may be between 3 and 10 equivalents relative to the molar amount of the compound of formula (II). The molar amount of base in step b1), when it is present, may be between 3 and 10 equivalents relative to the molar amount of the compound of formula (II).

The base in step b1), when it is present, may optionally be recovered and reused, i.e. recycled. In a particular embodiment, the method according to the invention further comprises, after step b1), a step of recovery of the base from step b1), preferably by distillation or evaporation at reduced pressure.

Step b2) makes it possible to convert said compound of formula (II′) obtained in step b1) to said compound of formula (III). Step b2) is carried out in the presence of a base. This base may be an oxygenated base such as a hydroxide (for example, sodium hydroxide or potassium hydroxide), an alcoholate (for example, sodium ethylate or sodium isopropylate) or a carbonate (for example, sodium carbonate).

Preferably, the base in step b2) is a hydroxide, in particular sodium hydroxide. The molar amount of base in step b2) may be between 4 and 25 equivalents relative to the molar amount of said compound of formula (II′).

In a particular embodiment, the reaction in step b2) is carried out in water or in a water/alcohol mixture. Examples of alcohol are in particular ethanol, propanol or butanol, preferably ethanol. The water may optionally contain one or more additives, which for example may be selected from glycol derivatives (for example ethylene glycol, diethylene glycol, or propylene glycol) and surfactants.

In one embodiment, the reaction in step b2) is carried out at a temperature between 80° C. and 110° C., preferably for a time between 1 h and 5 h.

In a particular embodiment, step b2) comprises formation of a mixture by adding a compound of formula (II′) as defined above to a solution of the base in water, and then stirring the resultant mixture at a temperature between 80° C. and 110° C., preferably for a time between 1 h and 5 h. The compound of formula (III) obtained in step b) of the method of the invention, and in particular in step b2), is typically obtained in the form of a precipitate. Step c) of the method of the invention comprises recovery of the compound of formula (III) obtained in step b). The compound of formula (III) may be isolated in step c) by any technique known by a person skilled in the art, for example by filtration.

The purity of the compound of formula (III) obtained by the method of the invention is advantageously greater than or equal to 90%, preferably greater than or equal to 95%, or even greater than or equal to 98%.

In a particular embodiment, the method comprises the following steps:
a) reacting a compound of formula (I) as defined above with glyoxylic acid in the presence of palladium on carbon and under a hydrogen atmosphere, in a polar solvent (such as a THF/water mixture), at a temperature between 35° C. and 120° C. (for example, between 35° C. and 65° C.), preferably for a time between 30 seconds and 8 h, to obtain a compound of formula (II) as defined above:
b1) reacting said compound of formula (II) with acetic anhydride, in the presence of a base, preferably an amine base, such as triethylamine, said reaction being carried out successively:
at a temperature between 10° C. and 40° C., preferably for a time between 15 minutes and 3 hours, and then
at a temperature between 70° C. and 110° C., preferably for a time between 15 minutes and 3 hours, to obtain a compound of formula (II′) as defined above:
b2) reacting said compound of formula (II′) with a base, preferably a hydroxide, such as sodium hydroxide, in water, at a temperature between 80° C. and 110° C., preferably for a time between 1 h and 5 h, to obtain a compound of formula (III) as defined above; and
c) recovery of said compound of formula (III).

In another particular embodiment, the method comprises the following steps:
a) reacting a compound of formula (I) as defined above with glyoxylic acid in the presence of palladium on carbon and under a hydrogen atmosphere, in a polar solvent (preferably, a THF/water mixture), at a temperature between 35° C. and 120° C. (for example, between 35° C. and 65° C.), preferably for a time between 30 seconds and 8 h, to obtain a compound of formula (II) as defined above:
a′) recovery of the palladium on carbon, preferably by filtration, and recovery of the polar solvent, preferably by distillation or evaporation at reduced pressure:
b1) reacting said compound of formula (II) with acetic anhydride, in the presence of a base, preferably an amine base, such as triethylamine, said reaction being carried out successively:
at a temperature between 10° C. and 40° C., preferably for a time between 15 minutes and 3 hours, and then
at a temperature between 70° C. and 110° C., preferably for a time between 15 minutes and 3 hours, to obtain a compound of formula (II′) as defined above:
b′) recovery of the amine base, preferably by distillation or evaporation at reduced pressure:
b2) reacting said compound of formula (II′) with a base, preferably a hydroxide, such as sodium hydroxide, in water, at a temperature between 80° C. and 110° C., preferably for a time between 1 h and 5 h, to obtain a compound of formula (III) as defined above; and
c) recovery of said compound of formula (III).

In another particular embodiment, the method according to the invention consists essentially of steps a), b1), b2) and c) as described in the present application. “Method consisting essentially of steps a), b1) and b2)” means a method consisting of reaction steps a), b1) and b2), step c) of recovery of the compound of formula (III), and that may further comprise one or more additional steps of conventional treatment, in particular at the end of each of the steps a), b1) and b2). These steps commonly used in organic synthesis are, for example, steps of washing, liquid-liquid extraction, filtration, purification or drying.

In a particular embodiment, the method consists essentially of the following steps: a) reacting a compound of formula (I) as defined above with glyoxylic acid in the presence of palladium on carbon and under a hydrogen atmosphere, in a polar solvent such as a THF/water mixture, at a temperature between 35° C. and 120° C. (for example, between 35° C. and 65° C.), preferably for a time between 30 seconds and 8 h, to obtain a compound of formula (II) as defined above:
b1) reacting said compound of formula (II) with acetic anhydride, in the presence of a base, preferably an amine base, such as triethylamine, said reaction being carried out successively:
at a temperature between 10° C. and 40° C., preferably for a time between 15 minutes and 3 hours, and then
at a temperature between 70° C. and 110° C., preferably for a time between 15 minutes and 3 hours, to obtain a compound of formula (II′) as defined above:
b2) reacting said compound of formula (II′) with a base, preferably a hydroxide, such as sodium hydroxide, in water, at a temperature between 80° C. and 110° C., preferably for a time between 1 h and 5 h, to obtain a compound of formula (III) as defined above; and
c) recovery of said compound of formula (III).

A preferred aim of the present invention is a method of preparing indigo comprising the following steps:
a) reacting anthranilic acid with glyoxylic acid under hydrogenation in the presence of a metal catalyst in a solvent, to obtain 2-(carboxymethylamino)benzoic acid:
b) conversion of 2-(carboxymethylamino)benzoic acid to indigo; and
c) recovery of the indigo.

Preferably, the metal catalyst is a palladium catalyst, preferably palladium on carbon.

In a particular embodiment, the method according to the invention further comprises a step of recovery of the metal catalyst, after step a), preferably by filtration.

In another particular embodiment, the method according to the invention further comprises, after step a), a step of recovery of the solvent from step a), preferably by distillation or evaporation at reduced pressure.

Preferably, the solvent from step a) is a polar solvent, such as THE or a THF/water mixture.

According to another particular embodiment, glyoxylic acid is obtained from ethanol or from biosourced glycolic acid.

According to another particular embodiment, the reaction in step a) is carried out at a temperature between 35° C. and 120° C., preferably for a time between 30 seconds and 8 h.

In particular, the amount of metal catalyst in step a), is between 0.0001% and 40% by weight, preferably between 5% and 25% by weight, relative to the weight of anthranilic acid, and the hydrogen pressure is between 1 and 30 bar, preferably between 5 and 20 bar.

In particular, step b) comprises:
b1) reacting 2-(carboxymethylamino)benzoic acid with acetic anhydride, to obtain 1-acetylindol-3-yl acetate; and
b2) reacting the 1-acetylindol-3-yl acetate with a base, to obtain indigo.

According to a preferred embodiment, the method according to the invention comprises the following steps:
a) reacting anthranilic acid with glyoxylic acid in the presence of palladium on carbon and under a hydrogen atmosphere, in a polar solvent, at a temperature between 35° C. and 120° C., preferably for a time between 30 seconds and 8 h, to obtain 2-(carboxymethylamino)benzoic acid
b1) reacting 2-(carboxymethylamino)benzoic acid with acetic anhydride, in the presence of a base, preferably an amine base, such as triethylamine, said reaction being carried out successively:
at a temperature between 10° C. and 40° C., preferably for a time between 15 minutes and 3 hours, and then
at a temperature between 70° C. and 110° C., preferably for a time between 15 minutes and 3 hours, to obtain 1-acetylindol-3-yl acetate:
b2) reacting the 1-acetylindol-3-yl acetate with a base, preferably a hydroxide, such as sodium hydroxide, in water, at a temperature between 80° C. and 110° C., preferably for a time between 1 h and 5 h, to obtain indigo; and
c) recovery of the indigo.

In a particular embodiment, the compound of formula (I), in particular anthranilic acid, is biosourced. “Biosourced” product or compound means a product or compound derived from renewable organic matter of microbial, plant, fungal, or animal origin, and more precisely microbial in the context of the present invention. As examples of bacteria producing anthranilic acid, we may mention, non-exhaustively, the Gram+ or Gram− bacteria, such as the bacteria of the genus Escherichia (Escherichia coli), Streptomyces, Bacillus, Cupridavidus, Corynebacterium, Mycobacterium, Kitasatospora, Luteipulveratus, Thermobifida, Thermomonospora, Frankia, Pseudonocardia, Saccharothrix, Kutzneria, Lentzea, Prauserella, Salinispora, Micromonospora, Actinoplanes, Catenulispora, Mycolicibacterium, Dietzia, Aeromicrobium, Nonomuraea, Blastococcus, Modestobacter, Saccharopolyspora, Amycolatopsis, Actinopolyspora, Acidimicrobium, Photorhabdus, Hoeflea, Azospirillum, Crinalium, and Cylindrospermum.

In a more particular embodiment, the compound of formula (I), in particular, anthranilic acid is produced by a recombinant host cell, said cell preferably being microbial. Examples of recombinant microbial host cells are described in International application WO 2022/090363 (PCT/EP2021/079927). According to a preferred embodiment of the invention, the recombinant microbial host cell is selected from Escherichia (Escherichia coli), Streptomyces, Bacillus, Cupridavidus, Corynebacterium, Mycobacterium, Kitasatospora, Luteipulveratus, Thermobifida, Thermomonospora, Frankia, Pseudonocardia, Saccharothrix, Kutzneria, Lentzea, Prauserella, Salinispora, Micromonospora, Actinoplanes, Catenulispora, Mycolicibacterium, Dietzia, Aeromicrobium, Nonomuraea, Blastococcus, Modestobacter, Saccharopolyspora, Amycolatopsis, Actinopolyspora, Acidimicrobium, Photorhabdus, Hoeflea, Azospirillum, Crinalium, and Cylindrospermum. According to an even more preferred embodiment, the recombinant microbial host cell is selected from Escherichia, Streptomyces, Corynebacterium and Bacillus, and even more preferably the recombinant microbial host cell is Escherichia coli.

### EXAMPLES

The invention will be better understood from the following examples, which are given purely for illustration and do not aim to limit the scope of the invention, which is defined by the accompanying claims.

### Example 1. Method of Preparing a Compound of Formula (III): Indigo

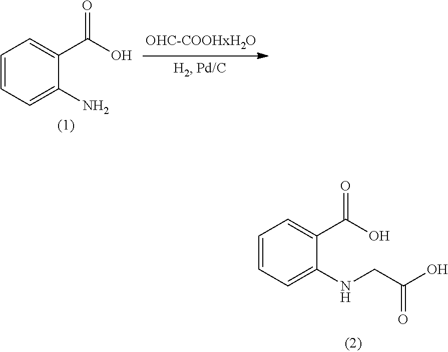 [SMILES: Nc1ccccc1C(=O)O ; O=C(O)CNc1ccccc1C(=O)O]

5 volumes/weight (also noted “v/w”, wherein 1 v/w has a value of 1 litre per kg) of THF is introduced, prior to loading, with stirring at 90 rpm, 1 kg of anthranilic acid and 0.1 kg of palladium on carbon (10% by weight of palladium, 50% moisture) in the reactor. The mixture is then pressurized to 10 bar of hydrogen and raised to 50° C. before introducing, in 30 min, 1.3 v/w of an aqueous solution at 50% of glyoxylic acid (1.2 molar equivalent).

The reaction mixture is then heated at 50° C. and stirred at 90 rpm for an additional 3.5 h. If the technical conditions allow regular sampling, the progress of the reaction may be monitored by HPLC.

When conversion is sufficient, >97%, the reaction is stopped and filtered on Celite (1 v/w) to remove the particles of palladium. The solvent is then evaporated to obtain 1.3 kg of 2-(carboxymethylamino)benzoic acid (2), the purity of which is evaluated at 85% by NMR. The 15% of impurities present consist essentially of minerals.

1H NMR (400 MHZ, DMSO): δ 7.78 (dd, J=8.1, 1.7 Hz, 1H), 7.29 (t, J=1.5 Hz, 1H), 6.54-6.51 (m, 2H), 3.75 (s, 2H) 13C NMR: (101 MHz, DMSO) δ 172.6, 170.9, 149.8, 132.9, 131.8, 114.0, 113.4, 111.0, 46.5 HPLC retention time: Anthranilic acid (1): 9.4 min; 2-(carboxymethylamino)benzoic acid (2): 13.1 min; main impurity: 8.6 min

Indigo was also prepared in the same experimental conditions as those described above except for the palladium on carbon, which was replaced either with palladium on aluminium oxide 0.5%, or nickel on aluminium oxide and silica (NiSat®: Ni/Al₂O₃—SiO₂ 54% Ni), or nickel on aluminium oxide (Ni/Al₂O₃ 21% Ni), or platinum on carbon.

Analytical data for HPLC:
Mobile phase: A: H₂O+0.1% of formic acid/B: Acetonitrile (HPLC purity)
Column: Kromasil KR5C18-25M (250×4.6 mm)
Gradient: to: 90/10; t=15 min: 50/50; t=20 min: 50/50; t=30 min: 90/10

Comparison of step a) of the method of the invention with the methods of the prior art (Table 1)

TABLE 1

| | | Yield | | Number | |
|---|---|---|---|---|---|
| | Reaction conditions | (%) | Waste products | of steps | Toxicity |
| Step a) of the | Glyoxylic acid | 97 | Wash water | 1 | — |
| method of the | Palladium on | | | | |
| invention | carbon + hydrogen | | | | |
| Step a) of the | Glyoxylic acid | 94 | Wash water | 1 | — |
| method of the | Palladium on | | | | |
| invention | aluminium oxide + | | | | |
| | hydrogen | | | | |
| Step a) of the | Glyoxylic acid | 90 | Wash water | 1 | — |
| method of the | NiSat + hydrogen | | | | |
| invention | | | | | |
| Step a) of the | Glyoxylic acid | 96 | Wash water | 1 | — |
| method of the | Nickel on | | | | |
| invention | aluminium oxide + | | | | |
| | hydrogen | | | | |
| Step a) of the | Glyoxylic acid | 97 | Wash water | 1 | — |
| method of the | Platinum on | | | | |
| invention | carbon + hydrogen | | | | |
| US2008/051426 | Chloroacetic acid | 80 | Basic water | 1 | Chloroacetic |
| | Na₂CO₃ | | contaminated with | | acid |
| | | | chloroacetic acid | | |
| Lai et al. ¹ | Chloroacetic acid | 90 | Reaction water and | 1 | Chloroacetic |
| | Na₂CO₃ + NaOH | | precipitation water | | acid |
| Koeppe et al. ² | Ethyl glyoxylate | 98 | Methanol, | 2 | NaBH₃CN |
| | NaBH₃CN in excess | | saponification water, | | |
| | | | NaBH₃CN residues | | |

¹ Helvetica Chimica Acta 2008, 91, 1975-1983
² ChemPhotoChem 2019, 3, 613-618

In contrast to the method of Lai et al. and that described in US 2008/051426, step a) of the method of the invention gives 2-(carboxymethylamino)benzoic acid with an excellent yield, using a non-toxic reactant, namely glyoxylic acid.

Access to this key intermediate is achieved in a single step, in contrast to the method of Koeppe et al., which uses saponification after coupling of anthranilic acid and ethyl glyoxylate. Finally, step a) generates a minimum of waste, as it uses a catalyst that is recyclable. It is also possible to recycle the used solvent, whereas the wastewaters in the methods of the prior art are contaminated with toxic reactants.

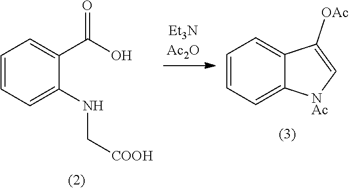

1-Acetylindol-3-yl acetate (3) can be formed in a “one-pot” reaction in the presence of 2-(carboxymethylamino)benzoic acid (2) (1 kg), acetic anhydride (2.15 kg or 2.0 L, or 5 equivalents) and triethylamine (2.1 kg or 2.8 L, or 5 equivalents). The reaction is first maintained at room temperature for 30 minutes to protect the amine. Then the reaction mixture is heated at 90° C. for 30 minutes for cyclization and protection of the hydroxyl.

After HPLC analysis confirming conversion greater than 95%, a portion (about 25%) of the volatile products may be evaporated at reduced pressure (40° C., 150 mbar) to recycle the triethylamine. 10 litres of water is then added in order to precipitate the product. The product is then filtered (25 microns), washed with 10 litres of water and then dried in a stove at 80° C., obtaining about 1.01 kg (91% of the expected weight) of powder. The purity of the product (3) thus obtained is determined by NMR analysis or HPLC (83%) in order to adjust the proportions of reactants for the next reactions. (measured yield: 75%).

1H NMR (400 MHZ, MeOD) δ 8.39 (d, J=8.3 Hz, 1H), 7.80 (s, 1H), 7.57-7.48 (m, 1H), 7.36 (ddd, J=8.5, 7.2, 1.4 Hz, 1H), 7.29 (td, J=7.5, 1.0 Hz, 1H), 2.61 (s, 3H), 2.37 (s, 3H).

HPLC retention time: Impurity (mono-acetylated product): 9.2 min: 1-acetylindol-3-yl acetate (3): 7.3 min

Analytical data for HPLC:
Mobile phase: A: H₂O+0.1% of formic acid/B: Acetonitrile (HPLC quality)
Column: Kromasil KR5C18-25M (250×4.6 mm)
Gradient: to: 50/50: t=15 min: 5/95: t=23 min: 5/95: t=25 min: 90/10; t=30 min: 50/50

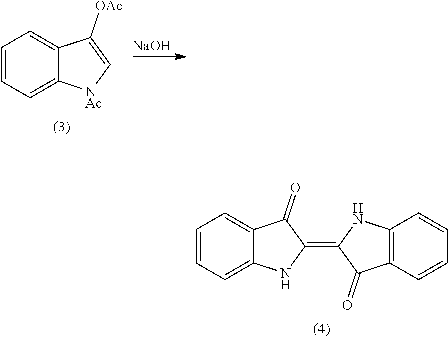

Preparation of indigo (4) starting from 1-acetylindol-3-yl acetate (3) (1 kg) takes place in an aqueous solution of sodium hydroxide (2.95 kg of sodium hydroxide in 23.6 L of solution, or 16 equivalents). First, the reaction mixture is heated under reflux for two hours, and then this mixture is left at room temperature with stirring for 5 hours. The blue precipitate thus formed is filtered (cutoff threshold 10 microns), and then washed with water (10 volumes) and with ethanol (10 volumes). The product is then dried in the stove (80° C.), obtaining 0.42 kg of indigo (Yield: 69%).

The purity of the final product is determined by UV spectroscopy. The purity of the indigo obtained is greater than 95%.

1H NMR (400 MHZ, DMSO) δ 10.50 (s, 2H), 7.61 (d, J=7.7 Hz, 2H), 7.51 (ddd, J=8.3, 7.1, 1.3 Hz, 2H), 7.33 (d, J=8.2 Hz, 2H), 6.95 (t, J=7.3 Hz, 2H).

1. A method of preparing a compound of formula (III): 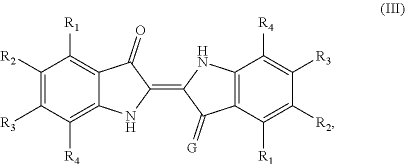
in which each of R₁, R₂, R₃ and R₄ is independently a hydrogen, a halogen, —CN, —NO₂, —C(O)H, —SO₃H, —CO₂H, —SO₃⁻M₁⁺, —CO₂⁻M₂⁺, —SO₃R₅, —CO₂R₆, —C(O)R₇ or —OR₈,
each of M₁⁺ and M₂⁺ being independently a cation,
each of R₅, R₆, R₇, and R₈ being independently a C₁-C₆ aliphatic group or an aryl;
the method comprising the following steps:
a) reacting a compound of formula (I): 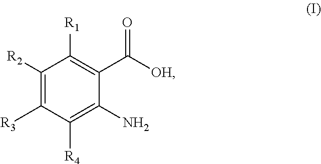
in which each of R₁, R₂, R₃ and R₄ is as defined above,
with glyoxylic acid under hydrogenation in the presence of a metal catalyst in a solvent, to obtain a compound of formula (II): 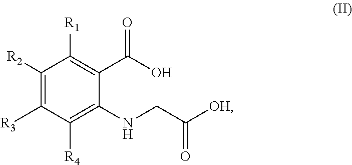
in which each of R₁, R₂, R₃ and R₄ is as defined above;
b) converting the compound of formula (II) to the compound of formula (III); and
c) recovering the compound of formula (III).

2. The method according to claim 1, wherein R₁, R₂, R₃, and R₄ are hydrogens.

3. The method according to claim 1, wherein the metal catalyst is a palladium, nickel, or platinum catalyst.

4. The method according to claim 1, wherein the metal catalyst is palladium on carbon, palladium on aluminium oxide, nickel on aluminium oxide, nickel on aluminium oxide and silica, or platinum on carbon.

5. The method according to claim 1, wherein the metal catalyst is palladium on carbon.

6. The method according to claim 1, wherein the method further comprises, after step a):
a step of recovering the metal catalyst; and/or
a step of recovering the solvent from step a).

7. The method according to claim 1, wherein the solvent in step a) is a polar solvent.

8. The method according to claim 1, wherein glyoxylic acid is obtained from ethanol or from biosourced glycolic acid.

9. The method according to claim 1, wherein the reaction in step a) is carried out at a temperature of from about 35° C. to about 120° C.

10. The method according to claim 1, wherein, in step a), the amount of metal catalyst is from about 0.0001% to about 40% by weight, relative to the weight of the compound of formula (I), and the hydrogen pressure is from about 1 bar to about 30 bar.

11. The method according to claim 1, wherein step b) comprises:
b1) reacting a compound of formula (II) as defined in claim 1 with acetic anhydride, to obtain a compound of formula (II′): 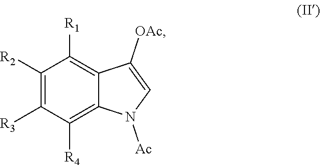
in which each of R₁, R₂, R₃ and R₄ is independently a hydrogen, a halogen, —CN, —NO₂, —C(O)H, —SO₃H, —CO₂H, —SO₃⁻M₁⁺, —CO₂⁻M₂⁺, —SO₃R₅, —CO₂R₆, —C(O) R₇ or —OR₈,
each of M₁⁺ and M₂⁺ being independently a cation,
each of R₅, R₆, R₇, and R₈ being independently a C₁-C₆ aliphatic group or an aryl,
preferably R₁, R₂, R₃ and R₄ are hydrogens; and
b2) reacting said compound of formula (II′) with a base, to obtain a compound of formula (III) as defined in claim 1.

12. The method according to claim 1, further comprising the following steps:
a) reacting a compound of formula (I) as defined in claim 1 with glyoxylic acid in the presence of palladium on carbon and under a hydrogen atmosphere, in a polar solvent, at a temperature of from about 35° C. to about 120° C., to obtain a compound of formula (II) as defined in claim 1;
b1) reacting the compound of formula (II) with acetic anhydride, in the presence of a base, the reaction being carried out successively:
at a temperature of from about 10° C. to about 40° C., and then
at a temperature of from about 70° C. to about 110° C., to obtain a compound of formula (II′) 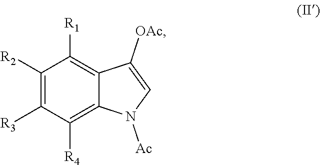
b2) reacting the compound of formula (II′) with a base in water, at a temperature of from about 80° C. to about 110° C. to obtain a compound of formula (III) as defined in claim 1; and
c) recovering the compound of formula (III).

13. The method according to claim 1, wherein the compound of formula (I), optionally anthranilic acid, is biosourced.

14. The method according to claim 1, wherein the compound of formula (I), optionally anthranilic acid, is produced by a recombinant host cell optionally microbial.

15. The method according to claim 14, wherein the recombinant microbial host cell is selected from the group consisting of Escherichia (Escherichia coli), Streptomyces, Bacillus, Cupridavidus, Corynebacterium, Mycobacterium, Kitasatospora, Luteipulveratus, Thermobifida, Thermomonospora, Frankia, Pseudonocardia, Saccharothrix, Kutzneria, Lentzea, Prauserella, Salinispora, Micromonospora, Actinoplanes, Catenulispora, Mycolicibacterium, Dietzia, Aeromicrobium, Nonomuraea, Blastococcus, Modestobacter, Saccharopolyspora, Amycolatopsis, Actinopolyspora, Acidimicrobium, Photorhabdus, Hoeflea, Azospirillum, Crinalium, and Cylindrospermum, preferably selected from Escherichia, Streptomyces, Corynebacterium and Bacillus.

16. The method according to claim 6, wherein the metal catalyst is recovered by filtration.

17. The method according to claim 6, wherein the solvent from step a) is recovered by distillation or evaporation at reduced pressure.

18. The method according to claim 7, wherein polar solvent is THF or a THF/water mixture.

19. The method according to claim 9, wherein the reaction in step a) is carried out for a duration of from about 30 seconds to about 8 hours.

20. The method according to claim 10, wherein the amount of metal catalyst is from about 5% to about 25% by weight.

21. The method according to claim 10, wherein the hydrogen pressure is from about 5 bar to about 20 bar.
